(19) United States
(12) Patent Application Publication
(10) Pub. No.: US 2014/0347071 A1
(43) Pub. Date: Nov. 27, 2014

(54) DEVICE AND METHOD FOR ASSESSING THE DEGRADATION OF THE INSULATION OF AN OIL-INSULATED TRANSFORMER

(71) Applicant: TECHIMP TECHNOLOGIES S.R.L., BOLOGNA (IT)

(72) Inventors: Gian Carlo Montanari, Casalecchio Di Reno (BO) (IT); Matteo Fattori, Cesena (FC) (IT); Stefano Serra, San Vittore Olona (MI) (IT)

(21) Appl. No.: 14/355,992

(22) PCT Filed: Nov. 14, 2012
(86) PCT No.: PCT/IB2012/056408

§ 371 Date: May 2, 2014

(30) Foreign Application Priority Data
Nov. 23, 2011 (IT) BO2011A000671

(51) Int. Cl. G01R 31/12; G01N 17/00

(52) U.S. Cl. CPC G01R 31/1281; G01N 17/00
USPC 324/553

(57) ABSTRACT

A device (1) for assessing the corrosion of the transformer windings in an oil-insulated transformer comprising a copper conductor (2) immersed in the oil. The change of the resistance of the copper conductor (2) over time is measured, e.g. using a Wheatstone bridge (2, 12a, 12b, 12c), and used to assess the degradation of the copper exposed to the insulation oil. The temperature of the copper conductor can be controlled by an heating element and a temperature sensor.

### TECHNICAL FIELD

This invention relates to a device and a method for assessing the degradation of the insulation of an oil-insulated transformer.

### BACKGROUND ART

It is known that the oil inside transformers (of the type used for high and medium voltages) contains sulphur, in the form of molecules such as, for example, benzyls, phenyls or other organic molecules (in particular dibenzyl disulphide, or DBDS).

This sulphur is quite corrosive to the copper the transformer windings are made of; indeed the sulphur bonds with the copper atoms of the windings, causing the copper to come away from the windings.

The copper atoms which react with the sulphur and come away from the windings settle on the transformer insulation paper, forming a layer of copper sulphide which, over time, causes the gradual and inexorable degradation of the paper insulation.

In time, this phenomenon degrades the insulation properties of the paper, leading to transformer overheating and even to extreme situations where short circuiting across the windings may cause the transformer to explode.

According to common practice in the prior art, transformer oil samples are taken at predetermined intervals for laboratory tests to measure the corrosiveness of the oil (according to well-defined standards).

This method is complex, particularly expensive and, what is more, not applicable in real time.

Also, this method provides an indication of the corrosiveness of the oil in the transformer but not of the state of corrosion of the windings.

Indeed, it should be noted that this method is not very reliable and of little significance for indicating the state of corrosion of the windings because it takes into account only the momentary state of the oil (which may have been recently changed and hence in excellent conditions, whereas the windings may be very worn).

Further, the corrosiveness of the oil is strongly dependent also on the temperature of the copper of the windings. Thus, this type of measurement is of even less significance as an indication of the degradation of the windings because it does not take into account the transformer load conditions and is influenced by the external temperature (in effect, the oil establishes a heat exchange relationship with the environment surrounding it).

Patent documents JP57207309 and U.S. Pat. No. 4,675,662 disclose systems for detecting the oil corrosion in a transformer; however, said systems are not precise, from a diagnostic point of view, because their measure is representative of the sulphur concentration, rather than the degradation of the insulation.

### DISCLOSURE OF THE INVENTION

The aim of this invention is to provide a device and a method for assessing the degradation of an oil-insulated transformer and which are at once simple, inexpensive and precise.

Another aim of the invention is to allow assessment of the state of degradation of a transformer in a particularly precise and accurate manner.

These aims are fully achieved by the device according to the invention as characterized in the appended claims.

In particular, the device for assessing the degradation of the insulation of an oil-insulated transformer due to the corrosion of the transformer windings comprises: a structure comprising a first portion and a second portion, the structure being configured to be attachable to the transformer in such a way that the first portion is outside the transformer and the second portion is inside the transformer and in contact with the oil;
an active element made of copper, defining a conductive path and connected to the second portion to be operatively in contact with the oil;
electrical contacts electrically connected to the active element at the terminals of the conductive path to allow reading of a value representing the resistance of the active element when the structure is attached to the transformer.

Preferably, the device comprises heating means to heat up the said active element. Preferably, the device comprises control means for driving the heating means.

Preferably, the control means are programmed for driving the heating means at a temperature variable over time, as a function of the load of the transformer.

These aims are also achieved by the method according to the invention as characterized in the appended claims.

In particular, the method for assessing the degradation of the insulation of an oil-insulated transformer due to the corrosion of the transformer windings comprises the following steps:
preparing a structure comprising a copper active element defining a conductive path;
coupling the structure to the transformer in such a way that the active element is in contact with the oil;
keeping the active element immersed in the oil at least for a predetermined interval of time between a starting instant and a final instant;
measuring a value representing the resistance of the active element at the final instant;
comparing the value measured at the final instant with a value representing the resistance of the active element at the initial instant in order to obtain an indication of the degradation.

### BRIEF DESCRIPTION OF DRAWINGS

This and other features of the invention will become more apparent from the following description of a preferred, non-limiting example embodiment of it, with reference to the accompanying drawings, in which:

FIG. 1 shows a schematic view of the diagnostic apparatus according to the invention;

FIG. 2 shows a schematic view of a detail of one embodiment of a device according to the invention;

FIG. 3 shows a schematic view of a further embodiment of the device according to the invention;

FIG. 4 shows a schematic view of a detail of a further embodiment of the device according to the invention.

### DETAILED DESCRIPTION OF PREFERRED EMBODIMENTS OF THE INVENTION

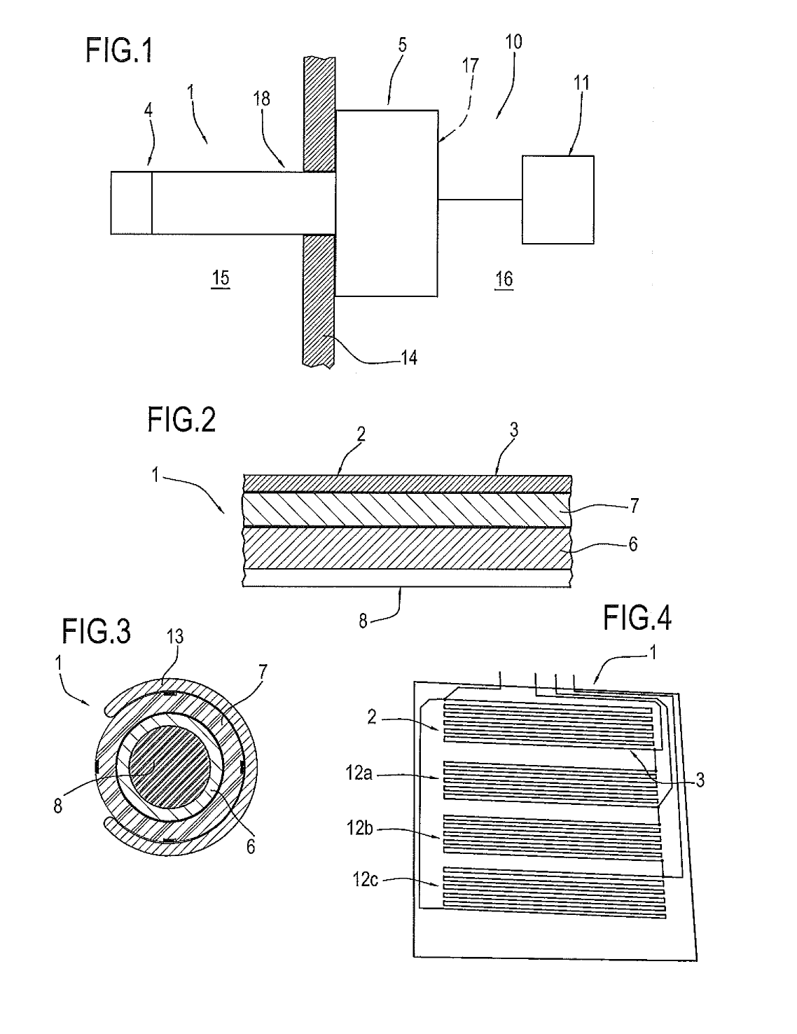

The numeral 1 in FIG. 1 denotes a device for assessing the degradation of the insulation of an oil-insulated transformer due to the corrosion of the transformer windings. In the description which follows, the device 1 is also referred to as probe 1.

The generic terms “wear” and “state of degradation” of the transformer are used interchangeably to denote the insulation conditions of the transformer windings.

It should be noted that, as mentioned above, the wear of the transformer windings due to the action of sulphur molecules causes the copper atoms to come away from the windings and to settle on the transformer insulation paper: this forms a layer of copper sulphide which, in time, causes a progressive and inexorable degradation of the paper insulation.

Thus, the state of degradation of the windings is correlated with the wear/corrosion of the transformer windings.

A generic transformer (not illustrated) which the sensor 1 can be used on is therefore a transformer whose windings are in an oil bath (insulated in oil) and comprises:
copper windings defining with the respective ferromagnetic cores the primary/secondary circuits of the transformer;
a container for the insulation oil which is in contact with the insulating paper of the windings.

The device 1 for measuring the state of degradation or wear of the transformer comprises an active element 2 made of metallic material (preferably and advantageously of copper).

Preferably, the element 2 is made of the same material as the windings are made of.

The copper element 2 is configured to be immersed in the oil in order to define a conductive path 3 in contact with the oil.

In other words, the copper element 2 is in contact with the oil in the transformer.

Hereinafter, the element 2 is referred to as copper element 2 or active element 2.

It should be noted that the fact that the copper element 2 is in contact with the oil means that the selfsame copper element 2 is corroded by the aggressive action of the molecules contained in the oil (in particular by the action of the sulphur or dibenzyl disulphide, that is, DBDS, or like substances) in the same way as the windings are (according to the phenomenon described above with reference to the prior art).

Thus, at the terminals of the conductive path 3, it is possible, in the manner clarified in more detail in the rest of this description, to sample a resistance signal s1 of the conductive path 3 representing the state of wear of the copper element 2 (the signal s1, as will become clearer as this description continues, in turn represents the state of wear of the transformer).

The copper element 2 extends principally along a longitudinal direction D so that the length of the conductive path 3 along that longitudinal direction is greater than that along other directions (for example, with respect to thickness and/or width).

In other words, the element 2 is made in such a way as to preferably define a conductive path with elongate shape and reduced cross section. Preferably, the copper element 2 is sized so that the ratio between the length of the copper element 2 along a direction of extension and the cross section of the copper element 2 is within the range from 10² cm⁻¹ to −10⁶ cm⁻¹; more preferably, the ratio is greater than 2.0 10⁴ cm⁻¹ and still more preferably, is approximately 2.0 10⁴ cm⁻¹.

It should be noted that, in other words, the tendency is to maximize the ratio between the length and the cross section of the copper element 2.

Following what is stated above, the copper element 2 is preferably in the form of a wire- or bar-like element whose thickness dimension is considerably smaller than its length dimension.

This gives the device the advantage of added sensitivity; in other words, under equal conditions of intensity/level of the factors which determine the wear of the copper element 2, the sensitivity of the resistance signal is greater (that is to say, there is a particularly high variation of the resistance signal, per unit of variation of the intensity/level of the factors which determine the wear of the copper element 2).

In particular, it should be noted that the copper element 2 is embodied in the form of a thin thickness copper track.

Still more preferably, the conductive path 3 has the winding shape of a coil.

Preferably, the conductive path has length greater than 200 mm and thickness less than 1 mm.

Preferably, the device comprises a structure 18 (hereinafter also referred to as body 18) having a first portion 5 and a second portion 4.

The first portion 5 and the second portion 4 might be made as a single part or as a plurality of elements to be coupled to each other.

The second portion 4 comprises the copper element 2 and, in use, is configured to be immersed in the oil.

The first portion 5 internally defines a housing (preferably a housing for the electronic circuitry for conditioning the measuring signal and/or for the electronic measuring circuitry) and, in use, is not in contact with the oil but is located outside the transformer.

It should be noted that the first portion 5 makes accessible from outside the transformer the measurement of a value representing the resistance of the copper element 2 and, more generally speaking, makes that value available from the outside.

According to the invention, the device 1 comprises electrical contacts 17 located in the first portion 5 and electrically connected to the element 2 (at the terminals of the conductive path 3) to allow reading, from outside the transformer, of a value representing the resistance of the element 2 when the structure 18 is attached to the transformer.

Preferably, the second portion 4 has a tubular shape (as illustrated in FIG. 3).

It should be noted that the copper element 2 may be fitted at various positions in the transformer: in the oil circulation pipes or inside the oil container or at the oil sampling port.

FIG. 2 schematically illustrates the end of the first portion where the copper element 2 is located.

It should be noted that the copper element 2 is mounted on a support 6 made of a thermally conductive material.

It should also be noted that the support 6 is not subject to corrosion by the corrosive agents in the oil.

The support 6 is made preferably of aluminium.

It should be noted that the copper element 2 is separated from the support 6 by a layer 7 of electrically insulating material.

Preferably, the layer 7 is a layer of polymeric or ceramic material.

The layer 7 is interposed between the copper element 2 and the support 6.

Still more preferably, the layer 7 is sized to allow transmission of heat between the support 6 and the copper element 2.

It should be noted that, preferably, the layer 7 allows good transmission of heat.

In a preferred embodiment, the layer 7 is in the form of a thin film.

The support 6 not only structurally supports the copper element 2 but also makes uniform the temperature of the components which are not in contact with the oil and which are located in the immediate vicinity of the copper element 2. This will become clearer as this description continues.

The device further comprises a heating element.

The heating element is preferably glued on the aluminium substrate; still more preferably, it is glued with two-sided adhesive resistant to high temperatures.

In the embodiment illustrated in FIG. 3, it should be noted that the copper element 2 comprises a layer of resistive rubber through which current passes in order to generate heat.

According to the invention, the heater defines means 8 for heating the copper element 2.

With reference to the circuit for measuring the resistance value of the conductive path 3 in contact with the oil, attention is drawn to the following.

The device 1 further comprises a Wheatstone bridge circuit (not illustrated) where one of the legs of the bridge is defined by the copper element 2.

The other three legs of the Wheatstone bridge are defined by further resistive element 12a, 12b, 12c which are not in contact with the oil, that is to say, they are insulated from the oil.

Preferably, the further resistive elements 12a, 12b, 12c have the same structure/configuration as the copper element 2.

The further resistive elements 12a, 12b, 12c are preferably covered by an insulating layer 13 which keeps them insulated from the oil.

The insulating layer 13 also improves the linearity of the further resistive elements 12a, 12b, 12c and to reduce the noise caused by the resistive elements 12a, 12b, 12c in the signal s1.

It should therefore be noted, more generally speaking, that one portion of the Wheatstone bridge is in contact with the oil while the other portion of the Wheatstone bridge is insulated from the oil.

Preferably, this type of bridge is a balanced bridge circuit. In other words, the resistance values of the four legs are equal to each other (plus or minus a certain tolerance). That means the device 1 is more simple to construct.

Thus defined is a diagnostic apparatus 10 for measuring the state of degradation of the transformer and comprising the device as described in the foregoing and means 9 for measuring the resistance of the copper element 2 and which are connected to the copper element 2 (through the contacts 17) in order to measure the resistance of the conductive path 3.

In the embodiment illustrated, the measuring means 9 are configured to measure a signal s1 which depends on the resistance of the conductive path 3 (in particular in the Wheatstone bridge configuration a signal is measured which indicates the unbalance of part of the bridge relative to the rest of it).

The measuring means 9 are preferably housed in the first portion 5.

In the preferred embodiment, the measuring means 9 comprise a signal conditioning module connected to the copper element 2 (and also to the further resistive elements 12a, 12b, 12c) to measure a signal dependent on the resistance of the copper element 2.

In particular, in the preferred embodiment, the conditioning module is connected to two corners of the Wheatstone bridge to receive a signal s1 dependent on the resistance value of the copper element 2. The Wheatstone bridge itself is powered at opposite corners of it.

The apparatus further comprises a temperature sensor for the copper element 2.

The temperature sensor is located in the second portion 4.

The temperature sensor is preferably glued (for example by an epoxy resin) to the support 6.

According to another aspect, the apparatus further comprises control means for driving the heating means 8 (switching them on/off).

The control means are preferably connected to the temperature sensor to receive a temperature signal and are configured to keep the copper element 2 at a predetermined temperature (in other words, they switch the heating means 8 on/off according to the value of the temperature signal).

Attention is drawn to the following with reference to the accompanying drawings.

FIG. 1 schematically illustrates an example of a device 1 where the second portion 4 is substantially tubular in shape, as illustrated in FIG. 3.

The following may be observed, starting from the centre: the heating element, the support 6, the electrically insulating layer, the resistive elements 3, 12a, 12b, 12c and the covering layer of the further resistive elements 12a, 12b, 12c.

In FIG. 1, the numeral 15 denotes the inside zone of the transformer (where the oil is), the numeral 14, the wall of the transformer, and the numeral 16, the outside of the transformer.

FIGS. 2 and 4 illustrate a variant embodiment of the device 1 where the support 6 and the electrically insulating layer are in the form of a plate: clearly shown are the copper element 2 (which the cross section of FIG. 2 refers to) and the further resistive elements 12a, 12b, 12c.

Below is a description of how the invention works, by way of example. In the description below, the following definitions are used:
the corrosion rate (Ca) of the windings is the rate at which the windings are corroded by the corrosive agents in the oil;
the corrosion rate (Cb) of the conductive path 3 is the rate at which the conductive path 3 of the copper element 2 is corroded by the corrosive agents in the oil;
the quantity of copper (Qa) in the windings is the quantity of copper remaining in the transformer windings, that is, the copper not removed by the corrosive agents in the oil;
the quantity of copper (Qb) in the conductive path 3 of the copper element 2 is the quantity of copper remaining in the conductive path 3, that is, the copper not removed by the corrosive agents in the oil;
the concentration level (z) of the corrosive agents in the oil is the concentration of the corrosive agents (for example, sulphur) which react with copper in the oil.

During normal use, the copper element 2 of the device 1 is immersed in, and in contact with, the oil; more specifically, in the preferred embodiment, the copper element 2 is exposed to the oil, while the remaining resistive elements 12a, 12b, 12c of the other legs of the Wheatstone bridge are insulated from the oil.

It should also be noted that the signal s1 made available by the device 1 is, in the preferred embodiment, a signal which depends not only on the resistance of the copper element 2 but also on that of the further resistive elements which make up the Wheatstone bridge.

It should therefore be noted that the signal s1 made available is proportional to the actual size of the conductive path 3 of the element 2 (in particular of the cross section of the path 3 of the copper element 2) that is to say, it is proportional to the quantity of copper in the copper element 2.

The variation of the signal s1 over time (and more specifically, the variation of the resistance value of the conductive path 3) depends on the corrosion rate Cb.

Generally speaking, the corrosion rate of a copper part, for example, of the copper element 2, depends on two factors: the concentration of the sulphur in the oil and the temperature of the copper.

In particular, the corrosion rate Cb of the copper element 2, generally speaking depends on two factors: the temperature of the copper element 2 and the concentration level Z of the corrosive agents in the oil (the term corrosive agents meaning sulphur, DBDS or other molecules).

In a first operating mode, described below, the copper element 2 is kept at a predetermined temperature which is constant over time: according to this mode, the resistance value of the copper element 2 represents the variation of the concentration of the corrosive agents in the oil.

It should be noted that in this mode, preferably, the temperature is measured (using the temperature sensor described above) near the copper element 2 and the heating element is switched on/off in such a way as to keep the temperature constant.

In other words, the temperature of the copper element 2 is controlled (preferably by a closed loop system for greater precision).

The measure of the variation in the resistance of the copper element 2 is directly representative of the corrosion rate Cb of the copper element 2.

It follows that if the temperature of the copper element 2 is kept constant, the measure of the variation in the resistance of the copper element 2 in a predetermined interval of time is a function of the concentration level Z of the sulphur in the oil over time.

Indeed, it should be noted that as the sulphur concentration in the oil increases, so the copper element 2 is corroded at a higher corrosion rate Cb, which in turn causes a greater variation in the resistance of the conductive path 3 over time in a predetermined time interval.

It should be noted that to obtain the measure of the quantity Qb of copper on the copper element 2 at a given instant, it is necessary to integrate the corrosion rate Cb over time.

This may be done by running a set of instructions in a processor (denoted by the reference numeral 11) which may or may not form part of the measuring apparatus 10.

It should be noted, however, that the value of the corrosion rate Cb does not represent the amount of corrosion of the windings (corrosion rate Ca) because, on account of the thermal effect of the current flowing through the windings, the temperature at the windings may be higher than that at the element 2 of the device 1 (which is usually not located in the same zone as the winding) and hence may have followed a different pattern over time.

Thus, to obtain an effective indication of the state of wear of the windings (and hence of the degradation of the transformer insulation) it is necessary to apply to the corrosion rate Cb (or more generally speaking, to the curve of the corrosion rates Cb) a mathematical “corrective” function which takes into account the difference between the temperature of the windings and the temperature of the copper element 2.

In other words, for example, if the temperature of the windings for a chosen period of time is greater than that of the copper element 2, the corrosion rate Ca of the windings is certainly greater than the corrosion rate Cb of the element 2; on the other hand, if the temperature of the windings for a chosen period of time is less than that of the copper element 2, it means the corrosion rate Ca of the windings is less than the corrosion rate Cb of the copper element 2.

It should be noted, therefore, that to estimate the extent of the corrosion of the windings, it is possible to apply to the values of the signal relating to the resistance of the conductive path 3 suitable corrective coefficients, that is, a mathematical function taking into account the transformer load; the transformer load may be measured directly or indirectly from the mains data or by measuring the temperature of the windings.

The quantity of copper Qa in the windings is given by the integral of the corrosion rate Ca of the windings over time.

Advantageously, this measuring mode may be applied in real time.

It should also be noted that according to this mode, it is possible to derive a measure of the quantity Qa of copper in the transformer in a particularly quick and easy manner without having to stop the transformer (for example to take an oil sample to be tested in the laboratory).

According to a second measuring mode, the state of wear/degradation of the transformer can be measured directly by measuring the resistance of the conductive path 3 of the copper element 2.

It has been observed that the variation in the resistance of the copper element 2 is directly representative of the corrosion of the copper windings by sulphur if the copper element 2 is kept at the same temperature as the windings.

It has been observed that the temperature of the transformer windings depends on the transformer load.

This mode comprises directly measuring the transformer load or, more preferably, the temperature of the windings (using a further temperature sensor to measure the temperature near the windings).

Thus, according to this mode, the copper element 2 is heated by the heating means 8 in order to keep it at the same temperature as the copper windings.

In particular, in the preferred embodiment where the copper element 2 forms part of a Wheatstone bridge, all the resistive elements forming part of the bridge are heated in such a way that the Wheatstone bridge is not unbalanced by differences of temperature between one leg and another but is unbalanced only by the variation in the resistance of the copper element 2.

The support 6 made of highly thermally conductive material in contact with the heating element advantageously makes it possible to bring the temperatures of all the resistive elements 3, 12a, 12b, 12c of the device 1 into line with each other.

It should be noted that keeping the copper element 2 (and the further resistive elements if a Wheatstone bridge configuration is adopted) at the same temperature as the windings means that the variation of the resistance signal of the copper element 2 represents the corrosion rate Ca of the copper windings of the transformer.

In other words, the element 2 is corroded at the same rate as the windings (Cb=Ca) and, hence, at any instant, the resistance signal s1 of the conductive path 3 represents the state of wear/corrosion of the copper windings, that is to say, the state of degradation of the transformer. In this mode, too, the signal s1 can be integrated over time to determine the actual state of the copper windings (in particular to determine the quantity of copper Qa in the windings).

Also defined according to the invention is a method for assessing the degradation of the insulation of an oil-insulated transformer due to the corrosion of the transformer windings, comprising the following steps:
preparing a structure 18 comprising a copper active element 2 defining a conductive path 3;
coupling the structure 18 to the transformer in such a way that the active element 2 is in contact with the oil;
keeping the active element 2 immersed in the oil at least for a predetermined interval of time between a starting instant and a final instant;
measuring a value representing the resistance of the active element 2 at the final instant;
comparing the value measured at the final instant with a value representing the resistance of the active element 2 at the initial instant in order to obtain an indication of the degradation.

The device 1 of this invention may be easily installed on new transformers but also mounted in an oil bath in existing types of transformers.

Also defined according to the invention is a further mode of measuring the state of degradation of a transformer particularly suitable for retro-fitting on transformers which are already in operation.

In this method, the device 1 for measuring a resistance value of the copper element 2 is fitted in an oil bath in the transformer.

The device is kept immersed in the oil bath for a predetermined period of time.

Preferably, the copper element 2 is kept at a very high temperature for the predetermined period of time, so as to accelerate the corrosion process (that is, it is heated by the heating means 8).

Next, after a chosen time interval has elapsed, the resistance of the copper element 2 is measured again.

It should be noted that the comparison of (that is, the difference between) the two resistance values can be used to derive the corrosion rate Cb of the copper element 2. From the corrosion rate Cb, if the temperature at which the copper element 2 has been kept is known, it is possible to derive the concentration (Z) of the corrosive agents in the oil.

This is a spot measuring system and not a continuous measuring system. According to a variant, it is possible to estimate the temperature curve of the windings in the period of time preceding the measurement (for example, by analysing the load data of the power mains) and to estimate a curve of the concentration level Z of the corrosive agents in the oil.

Starting with these input data, the above mentioned two measurements of the resistance of the copper element 2 are performed in the predetermined time interval. These two measurements are used, together with the estimate of the winding temperature in the preceding period and of the concentration level Z of the corrosive agents in the oil, to derive the quantity Qa of copper in the windings.

The purpose of this mode of measuring winding wear is to obtain an estimate of the quantity Qa of copper in the windings as if the device 1 were present and operational from the start of the working life of the transformer.

It should be noted that the device 1 according to the invention can be used, in accordance with what is described above (preferably in real time but also in a spot system), to perform two types of measurement:
a measurement of the variation of the concentration of sulphur (or DBDS) in the oil, or more generally speaking, of substances which corrode copper (corrosive agents);
a measurement of the state of wear of the transformer insulation.

It should also be noted that the device 1 is configured to be installed directly in the oil sampling valve, thus allowing it to be fitted directly also in existing types of transformers.

Attention is also drawn to the following with regard to temperature.

As is known, the value of resistance depends also on temperature.

To compensate for the variation of the resistance due only to temperature, in the method/apparatus described above, the coefficients are derived experimentally.

It should also be noted that another advantage of the Wheatstone bridge structure is the improved immunity of the signal s1 with respect to temperature variations.

1. A method for assessing the degradation of the insulation of an oil-insulated transformer due to the corrosion of the transformer windings, characterized in that it comprises the following steps:
preparing a structure (18) comprising a copper active element (2) defining a conductive path (3);
coupling the structure (18) to the transformer in such a way that the active element (2) is in contact with the oil;
keeping the active element (2) immersed in the oil at least for a predetermined interval of time between a starting instant and a final instant;
measuring a value representing the resistance of the active element (2) at the final instant;
comparing the value measured at the final instant with a value representing the resistance of the active element (2) at the initial instant in order to obtain an indication of the degradation.

2. The method according to claim 1, comprising a step of heating the active element (2) in order to take it to a predetermined temperature.

3. The method according to claim 2, wherein the step of heating the active element (2) comprises keeping the active element (2) at a constant temperature in the predetermined interval of time.

4. The method according to claim 2, wherein the active element (2) is heated to a variable temperature according to a profile corresponding to a load profile of the transformer in the predetermined time interval.

5. A device (1) for assessing the degradation of the insulation of an oil-insulated transformer due to the corrosion of the transformer windings, characterized in that it comprises:
a structure (18) having a first portion (5) and a second portion (4), the structure (18) being configured to be attachable to the transformer in such a way that the first portion (5) is outside the transformer and the second portion (4) is inside the transformer and in contact with the oil;
an active element (2) made of copper, defining a conductive path (3) and connected to the second portion (4) to be operatively in contact with the oil;
electrical contacts (17) electrically connected to the element (2) at the terminals of the conductive path (3) to allow reading of a value representing the resistance of the element (2) when the structure (18) is attached to the transformer.

6. The device according to claim 5, wherein the electrical contacts (17) are located in the first portion (5) to allow reading of the value representing the resistance of the active element (2) from outside the transformer without removing the device.

7. The device according to claim 5 or 6, wherein the active element (2) is made in such a way as to define a conductive path with elongate shape and reduced cross section.

8. The device according to any of the claims from 5 to 7, wherein the active element (2) is located on an outside surface of the second portion (4), the outside surface being defined by an electrical insulating element.

9. The device according to any of the claims from 5 to 8, comprising heating means (8) operatively connected to the second portion (4) of the structure (18) in order to heat the active element (2) coupled thereto.

10. The device according to claim 9, wherein the heating means (8) comprise a heating element inside the second portion (4) of the structure (18) and a thermally conductive support (6) interposed between the heating element and the active element (2).

11. The device according to claim 10, comprising a layer (7) of insulating material interposed between the active element (2) and the support (6).

12. The device according to any of the claims from 9 to 11, comprising a temperature sensor configured to measure the temperature of the active element (2) and control means for driving the heating means (8), connected to the temperature sensor to receive a signal relating to the temperature of the active element (2) and programmed to take the active element (2) to a predetermined temperature.

13. The device according to any of the claims from 5 to 12, comprising three further copper elements (12a, 12b, 12c), connected to the electrical contacts (17) in such a way that the active element (2) defines one of the legs of a Wheatstone bridge connected to the electrical contacts (17).

14. The device according to any of the claims from 5 to 13, comprising means (9) for measuring the resistance of the active element (2), connected to the electrical contacts (17) to measure a signal representing the resistance of the conductive path (3), the measuring means (9) being positioned in a housing defined by the first portion (5) of the structure (18).

15. The device according to claim 14, comprising a processor programmed to process at least one resistance value measured for the active element (2) at least at the final instant of a predetermined interval of time during which the device has been kept coupled to the transformer, by comparing the value measured at the final instant with a value representing the resistance of the active element (2) at the starting instant in order to derive an indication of the degradation.

16. Use of a device according to any of the claims from 5 to 14 for assessing the degradation of the insulation of an oil-insulated transformer due to the corrosion of the transformer windings, comprising the following actions:
coupling the device (1) to the transformer in such a way that the active element (2) is in contact with the oil;
keeping the device (1) coupled to the transformer for a predetermined interval of time, between a starting instant and a final instant in such a way that the active element (2) remains immersed in the oil during the predetermined interval of time;
measuring a value representing the resistance of the active element (2) at the final instant;
comparing the value measured at the final instant with a value representing the resistance of the active element (2) at the initial instant in order to obtain an indication of the degradation.
